(19) United States
(12) Patent Application Publication
(10) Pub. No.: US 2025/0082553 A1
(43) Pub. Date: Mar. 13, 2025

(54) Tooth Surface Treating Agent and Tooth Surface Treating Kit

(71) Applicant: Nishio Co. Ltd., Chiba (JP)

(72) Inventors: Hidetoshi Nishio, Chiba (JP); Naoko Miki, Tokyo (JP)

(21) Appl. No.: 18/700,823

(22) PCT Filed: Sep. 28, 2022
(86) PCT No.: PCT/JP2022/036088

§ 371 Date: Apr. 12, 2024

(30) Foreign Application Priority Data
Oct. 13, 2021 (JP) 2021-168057

(51) Int. Cl. A61K 8/24; A61K 8/21; A61K 8/362; A61Q 11/00

(52) U.S. Cl. CPC A61K 8/24; A61K 8/21; A61K 8/362; A61Q 11/00; A61K 2800/884

(57) ABSTRACT

The present invention provides a tooth surface treatment agent and a tooth surface treatment kit to form a uniform, high-strength layer composed of apatite on tooth surface and to repair minute recesses on the tooth surface.

The tooth surface treatment agent includes: a liquid with a pH of 1.0 to 3.0 containing an organic acid; and hydroxyapatite particles with a particle diameter of 200 nm or less. The tooth surface treatment kit includes: a first treatment agent that is a liquid having a pH of 1.0 to 3.0 and containing an organic acid, which is accommodated in a first container; and a second treatment agent including hydroxyapatite particles having a particle diameter of 200 nm or less, which is accommodated in a second container different from the first container. The tooth treatment agent is prepared by mixing the first and second treatment agents, and is applied to the tooth surface.

### TECHNICAL FIELD

This invention relates to a tooth surface treatment agent and a tooth surface treatment kit for mammals including humans. More specifically, the invention relates to a tooth surface treatment agent and a tooth surface treatment kit for restoring tooth surfaces by forming a layer covering minute recesses on the tooth surface.

### BACKGROUND TECHNOLOGY

Dental plaque deposits on tooth enamel surface, and an acid produced by microorganisms in the plaque gradually dissolves the enamel (so-called demineralization). As this progresses, the tooth becomes decayed, which can be detected by naked eyes. Common dental treatment involves grinding away hard tissue of caries that can be detected by naked eyes, including the surrounding healthy areas, and filling the defect with various types of plastic, cement, or metal as a replacement for the missing portion. Micro fillers for filling fissures or small surface demineralization wounds in tooth enamel before they progress to caries are disclosed in patent document 1.

The micro filler disclosed in the patent document 1 is a dental micro filler in the form of powder, granule, solution (suspension) or paste consisting of hydroxyapatite and an auxiliary agent. Hydroxyapatite is used when it is pulverized into a size of 0.02 to 10 μm. The dental micro filler is rubbed into the teeth after plaque removal, using a fingertip, brush, stick, cloth or the like for at least 1 minute, preferably 3 minutes.

### PRIOR ART DOCUMENTS

### Patent Literature

Patent Document 1: U.S. Pat. No. 2,724,307

### SUMMARY OF THE INVENTION

### Problems to be Solved by the Invention

However, even if hydroxyapatite is finely milled into about 0.02 to 10 μm, hydroxyapatite particles will agglomerate if the dispersing medium is not configured to stabilize hydroxyapatite particles when made into a solution (suspension) or paste. When hydroxyapatite powder was applied directly to teeth or liquid or paste containing agglomerated hydroxyapatite particles was applied to teeth, the layer formed on the tooth surface would have problems in uniformity, strength and long-term durability.

The present invention was made in view of the above-mentioned problems, and its purpose is to provide a tooth surface treatment agent and a tooth surface treatment kit for forming a layer consisting of apatite with uniformity and high strength on the tooth surface and for restoring minute recesses on the tooth surface.

### Means for Solving Problems

The tooth surface treatment agent of the present invention is characterized in that it contains a liquid with a pH of 1.0 to 3.0, containing an organic acid and hydroxyapatite particles with a particle diameter of 200 nm or less.

According to the tooth surface treatment agent of the present invention, when applied to the tooth surface containing minute recesses, the surface treatment agent reacts on the tooth surface to form a layer covering the entire tooth including the minute recesses. The aforementioned layer formed is more uniform and stronger than those formed by conventional methods. Therefore, it is possible to ensure a restoration of tooth surfaces with excellent long-term durability.

Further, the tooth surface treatment kit according to the present invention includes a first treatment agent which is a liquid containing an organic acid and has a pH of 1.0 to 3.0, that is accommodated in a first container, and a second treatment agent including hydroxyapatite particles having a particle size of 200 nm or less, that is accommodated in a second container. A tooth surface treatment agent prepared by mixing together the first treatment agent and the second treatment agent is applied to the tooth surface.

According to the tooth surface treatment kit, when the tooth surface treatment agent prepared by mixing together the first and second treatment agents is applied to the tooth surface including the microscopic recesses, the surface treatment agents will react on the tooth surface to form a layer covering the entire tooth including the microscopic recesses. The aforementioned layer formed is more uniform and stronger than those formed by conventional methods. Therefore, a tooth surface restoration with an excellent long-term durability can be achieved. The tooth surface treatment kit also allows the tooth surface treatment agent to be prepared each time it is used. The tooth surface treatment agent prepared for each use has stable dispersion and dissolution of hydroxyapatite particles and a stable pH. In addition, the tooth surface treatment agent prepared each time of use can be prepared at a dental institution or the like, where the mixing amount of the first and second treatment agents can be determined according to conditions of teeth to be treated.

The kit of the present invention for treating tooth surfaces further includes a liquid fluoride treatment agent containing at least one of sodium fluoride, calcium fluoride, and stannous fluoride, which is accommodated in a third container separated from the first and second containers. After the tooth surface treatment agent applied to the tooth surface has been removed, the fluoride treatment agent is preferably applied to the tooth surface. By this configuration, the layer formed by the tooth surface treatment agent has an increased density, thickness, and strength. Thus, it is possible to ensure a restoration of tooth surfaces with even better long-term durability.

### Effect of the Invention

The tooth surface treatment agent and tooth surface treatment kit of the present invention can be used to treat tooth surface to form a uniform, high-strength layer of apatite on the tooth surfaces and restore the microscopic recesses on the tooth surfaces.

### BRIEF DESCRIPTION OF THE DRAWINGS

FIG. 1 is a schematic illustration of a tooth surface treatment method using a tooth surface treatment kit as an embodiment of the present invention.

FIG. 2 is a diagram for explaining the effect of tooth surface repair by using the tooth surface treatment kit according to an embodiment of the present invention. FIG. 2A is an electron microscope photograph (reflected electron image) of the enamel surface layer of the cleaned test tooth. FIG. 2B is an electron microscope photograph (reflected electron image) of the enamel surface layer of a test tooth treated with a tooth surface treatment agent and a fluoride treatment agent after cleaning.

### EMBODIMENTS FOR CARRYING OUT THE INVENTION

The tooth surface treatment agent and tooth surface treatment kit of the present invention, the tooth surface treatment method using the tooth surface treatment agent and tooth surface treatment kit of the present invention will all be described below.

The tooth surface treatment agent of the present invention is prepared by mixing a liquid first treatment agent of pH 1.0 to 3.0 containing organic acid and a second treatment agent of hydroxyapatite particles with a particle diameter of 200 nm or less, while the hydroxyapatite particles of the second treatment agent are dispersed and dissolved in the liquid first treatment agent. Here. “dispersed and dissolved” means that hydroxyapatite particles of the second treatment agent are dispersed as nanoparticles in the liquid first treatment agent or will become transparent with almost no agglomerated particles dissolved.

The tooth surface treatment agent of the present invention is prepared by mixing 0.01 g to 1 g of the second treatment agent into 1 ml of liquid first treatment agent. The pH of the tooth surface treatment agent is adjusted to 4.5 to 6.5.

Therefore, the organic acid contained in the rust treatment agent should have the function of being chemically or physically adsorbed on the surface of hydroxyapatite particles to prevent their agglomeration in the liquid, in addition to dissolving hydroxyapatite particles by the acid action. Carboxylic acid having two or more carboxy groups can be adsorbed on the surface of hydroxyapatite particles by virtue of chelate-formation ability for Ca²⁺ ion.

In addition to its function as a medium to dissolve and disperse hydroxyapatite particles in the second treatment agent, the first treatment agent has the function to dissolve and alter pellicle and coloring components attached to the tooth surface by the tooth surface treatment agent. In addition, the first treatment agent has the function of etching hydroxyapatite that constitutes the tooth surface to be treated. Therefore, the first treatment agent can be used as a cleansing agent to remove pellicle and coloring components attached to the tooth surface and to expose the tooth surface itself.

The organic acid contained in the liquid of the first treatment agent, which is also used to clean the tooth surface, should be an organic acid with reducing properties and/or chelate-forming ability for calcium ions. Citric acid, tartaric acid, malic acid, and ascorbic acid are examples of such organic acids. Reducing properties contribute to the decomposition of organic matter. The complexing ability to calcium ions promotes the acid etching action. The chelate forming ability for calcium ions will be adsorbed to the surface of hydroxyapatite particles and partially dissolved particles (hereinafter collectively referred to as “apatite nanoparticles”) of the second treatment agent, with the apatite nanoparticles being negatively charged. This suppresses the aggregation of apatite nanoparticles.

When the first treatment agent is also used to clean the tooth surface, it is necessary to ensure that the tooth surface is not excessively etched. If the excessive etching enlarges the fine recesses on the tooth surface, it will be difficult to cover the recesses with the tooth surface treatment agent. A low pH of the first treatment agent liquid requires strict control of the desired etching process time. The pH of the liquid of the first treatment agent was set between 1.0 and 3.0 so that the etching of the tooth surface can be properly controlled. Considering the treatment time in the dental clinic office. pH of 1.2 to 2.5 for the liquid of the first treatment agent is desirable. A pH of 1.5 to 2.0 for the liquid of the first treatment agent is particularly desirable. The concentration and pH of the organic acid are selected according to the staining conditions of the tooth surface. Citric acid is suitable as an organic acid for the first treatment agent, this is because citric acid, in addition to its reducing properties and chelate-forming ability for calcium ions, it has a pH of 1.5 to 2.0 when being prepared in an aqueous solution of 0.1 to 1 mol/l.

The liquid of the first treatment agent should be a high-viscosity liquid (gel-like) in view of an easiness when being applied. To make it a high viscosity liquid, the liquid of the first treatment agent can contain a thickening or gelling agent that is acidic, stable, and orally acceptable. The thickening agent or gelling agent also inhibits the aggregation of apatite nanoparticles. Examples of thickening agent or gelling agent that can be used are carboxymethyl cellulose (CMC) or its sodium salt for cellulosic polymers, but can be xanthan gum, psyllium seed gum, and tamarind seed gum for polysaccharide thickening agents.

The second treatment agent that is hydroxyapatite, is a powder prepared by grinding biological original materials such as eggshells or chemically prepared powders. The particle size of the hydroxyapatite is 200 nm or less (less than visible light wavelength). Particle size of 200 nm or less can cause rapid reaction with the components contained in the liquid of the second treatment agent. Particle diameters of 100 nm or less are more desirable. In addition, hydroxyapatite of biological origin is desirable because it contains organic matter and other substances derived from raw materials. As described below, biologically derived hydroxyapatite is superior in terms of the characteristics of the layer formed on the tooth surface by the tooth surface treatment agent. The reason for this is not clear, but it is understood that organic substances derived from raw materials or metals other than Ca have some effects.

The tooth surface treatment method using the tooth surface treatment agent of the present invention includes: a cleaning step in which the tooth surface is cleaned, and a tooth surface treatment step in which the tooth surface to be treated and has been cleaned in the cleaning step is treated with the tooth surface treatment agent. In the tooth surface treatment step, a layer consisting of crystals such as hydroxyapatite is formed on the tooth surface. The layer formed on the tooth surface covers (closes) the microscopic recesses on the tooth surface. The layer formed on the tooth surface has an excellent strength and acid resistance.

By virtue of the cleaning step, the tooth surface to be treated is cleaned so that no pellicle or coloring components (organic matter) remain before the tooth surface treatment step. Therefore, the layer formed on the tooth surface is also prevented from degradation caused by residual pellicle and coloring components (organic matter) in the recesses.

The cleaning step may be performed by carefully cleaning the surface of the teeth to be treated, by virtue of a combination of tooth brushing with a toothbrush, wiping with a wipe material, and cleaning with a liquid cleaning agent such as mouthwash. On the other hand, the cleaning step can be performed reliably and efficiently by using the first treatment agent as a cleansing agent. The cleaning step can be carried out specifically as follows. First, the first treatment agent serving as a cleansing agent is applied to the tooth surface to be treated. With the cleansing agent applied in such a condition, the tooth surface to be treated is left for a predetermined time. After the predetermined time, the cleansing agent applied to the tooth surface to be treated is removed by rinsing the tooth surface, such as by spraying water on the tooth surface to be treated. The predetermined time is selected in a range of 30 seconds to 5 minutes, depending on the condition of the teeth to be treated. Further, the predetermined time can be shortened by irradiating and heating the teeth with infrared rays.

The removal of the cleansing agent from the tooth surface should be preceded by wiping with a melamine foam (sponge made of melamine), followed by rinsing, such as spraying water on the tooth surface. The pellicle and coloring components are weakened in their adhesion to the tooth surface due to an action of the cleansing agent. In addition, melamine foam has a high ability to remove surface adhering substance by wiping. Therefore, wiping with melamine foam absorbs and removes the cleansing agent from the liquid in which the pellicle and coloring components are dissolved, and also strips off the pellicle and coloring components from the tooth surface to be treated.

The tooth surface treatment step can be specifically carried out as follows. Namely, the tooth surface treatment agent is applied to the tooth surface that has been cleaned in the cleaning step. With the tooth surface treatment agent applied, the tooth surface is left for a predetermined time. After the predetermined time has passed, the tooth surface treatment agent applied to the tooth surface is removed by rinsing the tooth surface with a jet of water or the like. The predetermined time is selected in the range of 1 to 10 minutes, depending on the condition of the teeth to be treated. The predetermined time can be shortened by irradiating and heating the teeth with infrared rays.

The removal of the tooth surface treatment agent on the surface of the tooth should be preceded by wiping the teeth with a wipe material before rinsing with a jet of water or the like on the tooth surface. If the wipe material is rubbed strongly during wiping, the layer consisting of crystals such as hydroxyapatite can be peeled off. Water-impregnated melamine foam is suitable as a wipe material because it can remove the tooth surface treatment agent (which is difficult to remove by only rinsing alone) if wiping is lightly done as if tracing the tooth surface. By wiping with water-impregnated melamine foam, it is possible to remove the excess Ca²⁺ ions on the tooth surface and the crystals that have not been settled on the tooth surface.

The tooth surface treatment agent of the present invention should be used in a stable state of dispersion and dissolution of hydroxyapatite particles and in a stable pH. Therefore, the tooth surface treatment kit of the present invention includes a first treatment agent of a liquid containing an organic acid with a pH of 1.0 to 3.0 (which is accommodated in a first container), and a second treatment agent of hydroxyapatite particles with a particle diameter of 200 nm or less (which is accommodated in a second container), so that the tooth surface treatment agent of the present invention can be prepared each time it is used. According to the tooth surface treatment kit, the tooth surface treatment agent can be prepared at a dental institution or other site, where the amount of mixing of the first and second treatment agents can be determined and prepared according to the teeth to be treated.

The tooth surface treatment method using the above-mentioned tooth surface treatment kit of the present invention includes: mixing a liquid first treatment agent containing an organic acid with a pH of 1.0 to 3.0 (accommodated in a first container) and a second treatment agent of hydroxyapatite particles with a particle diameter of 200 nm or less (accommodated in a second container). The method includes: a tooth surface treatment agent preparation step in which a tooth surface treatment agent is prepared by dispersing and dissolving hydroxyapatite particles of the second treatment agent in the liquid first treatment agent; a cleaning step in which the first treatment agent is used as a cleansing agent to clean the surface of the tooth to be treated; and a tooth surface treatment step in which the tooth surface treatment agent is applied to the tooth surface cleaned in the above-mentioned cleaning step. The cleaning step using the first treatment agent as the cleansing agent is as described above. The tooth surface treatment step of applying the tooth surface treatment agent is also as described above.

In order to increase the whiteness of teeth, and to increase the acid resistance of tooth structure, further to increase the strength of tooth surface, the tooth surface treatment method using the tooth surface treatment agent or the tooth surface treatment kit should include a fluoride treatment step that follows the tooth surface treatment step. The fluoride treatment step can be specifically carried out as follows. Namely, a fluoride treatment agent is applied to tooth surface on which a layer of hydroxyapatite or other crystals has been formed in the tooth surface treatment step. The tooth surface that has been coated with the fluoride treatment agent is left for a predetermined period of time. After the predetermined time has passed, the cleansing agent applied to the tooth surface is removed by rinsing the tooth surface, such as by spraying water on the tooth surface. The predetermined time is selected in the range of 30 seconds to 10 minutes, depending on the condition of the teeth to be treated. The predetermined time can be shortened by irradiating and heating the teeth with infrared rays.

The removal of the fluoride treatment agent from the tooth surface should be preceded by wiping the tooth surface with a wipe material before rinsing with a jet of water or the like on the tooth surface. If the wipe material is rubbed strongly during wiping, the layer consisting of crystals such as hydroxyapatite may be peeled off. Water-impregnated melamine foam is suitable as a wipe material because it can remove the fluoride treatment agent (which is difficult to remove by rinsing alone) by wiping lightly as if tracing the tooth surface. When wiping is performed using water-impregnated melamine foam, residual fluoride agent can be removed to the extent that no trace of the fluoride agent can be found even after drying.

The fluoride treatment agent is a liquid containing at least one of the following fluorides: sodium fluoride, calcium fluoride, and stannous fluoride. The concentration of the fluorides in the fluoride treatment agent is from 500 ppm to 10,000 ppm. The liquid of the fluoride treatment agent should be in gel form for an easy applying treatment. When the fluoride is sodium fluoride, the liquid of the fluoride treatment agent can be acidic and stable, and can contain an orally acceptable thickening or gelling agent. For example, among cellulose polymers it is possible to use carboxymethyl cellulose (CMC) or its sodium salt, and among polysaccharide thickeners it is possible to use xanthan gum, samarium seed gum and tamarind seed gum, all being used as thickeners or gelling agents for the fluoride treatment agent.

The kit of the present invention for tooth surface treatment should further include a liquid fluoride treatment agent containing at least one of sodium fluoride, calcium fluoride, and stannous fluoride, which is accommodated in a third container separated from the first and second containers.

### Examples

The first treatment agent, second treatment agent, and fluoride treatment agent included in the tooth surface treatment kit in this example are as follows. Details of the formulations of the first and second treatment agents are shown in Table 1. Comparative examples 1 to 2 in Table 1 have used phosphoric acid solution as the first treatment agent.
First treatment agent: gel-like liquid containing organic acid
Second treatment agent: hydroxyapatite powder
   Particle diameter: 50 nm or less
Fluoride treatment agent: gel-like liquid containing sodium fluoride
   Sodium fluoride: 9000 ppm

The tooth surface treatment agent used in this example was prepared by mixing the first treatment agent and the second treatment agent prior to the implementation of the tooth surface treatment method in this example. In examples 1 to 6 and comparative examples 1 to 2, the amount (g) of the second treatment agent listed in Table 1 was mixed with 1 ml of the first treatment agent.

TABLE 1

| | | examples | | | | | | comparative examples | |
|---|---|---|---|---|---|---|---|---|---|
| | | 1 | 2 | 3 | 4 | 5 | 6 | 1 | 2 |
| first | acid | 0.5 mol/l citric acid | | | 0.5 mol/l | 0.5 mol/l | 0.5 mol/l | 0.5 mol/l | |
| treatment | | adjust with hydrochloric acid or NaOH | | | malic acid | ascorbic acid | acetic acid | phosphoric acid | |
| agent | pH | 1.8 | 1.2 | 1.8 | 1.5 | 2.5 | 2.6 | 1.2 | 1.2 |
| second | biological | | 0.2 | | | | | | |
| treatment | origin | | | | | | | | |
| agent | biological | 0.05 | | | 0.05 | 0.02 | 0.02 | 0.2 | 0.02 |
| | origin | | | | | | | | |
| | chemical | | | 0.05 | | | | | |
| | synthesis | | | | | | | | |

| | | | | | | | | |
|---|---|---|---|---|---|---|---|---|
| dissolved, dispersed | ◯ | ◯ | ◯ | ◯ | ◯ | ◯ | X | ◯ |
| layer formation | ⊚ | ◯ | ◯ | ◯ | Δ | Δ | X | X |

The teeth of animals treated with phosphoric acid to form fine grooves on tooth surface and then treated by attaching coloring components (hereinafter sometimes referred to as “test teeth”) was treated with the combination of the first treatment agent and the second treatment agent in examples 1 to 6 and comparative examples 1 to 2. The state of dissolution and dispersion of the second treatment agent with respect to the first treatment agent was confirmed by naked eye. The formation of the layer by the tooth surface treatment agent was evaluated by electron microscope (reflected electron image).

“Dissolution/dispersion state” in Table 1 indicates the transparency of the dissolution/dispersion state of the second treatment agent with respect to the first treatment agent, when confirmed by naked eyes. “◯” indicates a state of transparency, and the “X” indicates a state of opacity. In examples 1 to 6, where the first treatment agent is a solution mainly composed of organic acid, hydroxyapatite of the second treatment agent is presumed to be dissolved or dispersed as nanoparticles. On the other hand, in comparative example 1, where the first treatment agent is a solution mainly composed of phosphoric acid, the amount of hydroxyapatite in the second treatment agent exceeds a range where it can be dissolved in phosphoric acid. This is understood to have caused agglomeration of hydroxyapatite and have caused an opacity.

At first, the test teeth were coated with the first treatment agent on the surface of the test teeth, left in that condition for 1 minute, and then rinsed with water (first step treatment). In examples 1 to 4 where the first treatment agent was an organic acid solution with a pH of less than 2, when the first stage treatment was performed, the coloring on the surface of the test teeth had disappeared. However, in examples 5 to 6 and comparative examples 1 to 2 with pH at 2 or above, the first-stage treatment needs to be repeated many times before the coloring on the surface of the test teeth disappears. It was judged that it is not practical to use a liquid having a pH of more than 3 as the first treatment agent.

Test teeth cleaned in the first treatment step were coated with a pre-prepared tooth surface treatment agent according to Table 1, and allowed to stand for 3 minutes, and then rinsed (second step treatment). After the second-stage treatment, the test teeth were dried, visually observed, and electron microscopic reflection electron image phots were taken.

It was confirmed that the layer formed in example 1 (which used a tooth surface treatment agent containing biologically derived hydroxyapatite and citric acid), was the most uniform and had some thickness. The layer formed in example 3 (which used chemically synthesized hydroxyapatite) was slightly thinner than the layer formed in example 1. It is assumed that the organic substances or metals other than Ca contained in the bio-derived hydroxyapatite are effectively working. Example 2 (in which the amount of hydroxyapatite contained in the tooth surface treatment agent was high) was slightly less uniform than the layer formed in example 1. The layer formed in example 4 was equivalent to the layer formed in example 3. It is inferred that example 4 was affected by the use of malic acid instead of citric acid and a fact that a pH is 1.5 (which was lower than that of example 1). The layers formed in examples 5 and 6 were thinner in thickness than the layer formed in example 4. It is understood that this is due to the fact that the amount of hydroxyapatite in examples 5 and 6 is smaller than that in examples 1 and 4, considering the dissolution and dispersibility of hydroxyapatite. In examples 5 and 6, the thickness is expected to be improved by extending the processing time.

On the other hand, the layer formed in comparative example 1 was significantly less uniform than those in examples 1 to 6. It is understood that the layer formed in comparative example 1 contains many agglomerated particles. In comparative example 2, the layer covering the fine grooves of the test teeth was not formed. In addition, in comparative example 2, pH did not rise above 4.5 even though almost all of the hydroxyapatite mixed in the phosphoric acid solution was dissolved or dispersed. In other words, it is understood that the tooth surface treatment agent of comparative example 2 does not satisfy the conditions for the formation of a layer covering the fine grooves of the test teeth.

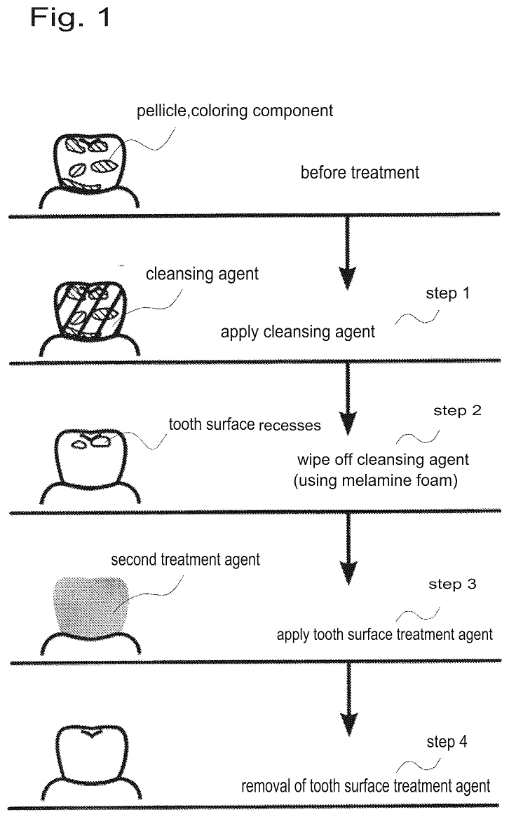

Next, the tooth surface treatment method using the tooth surface treatment kit described above will be described according to FIG. 1. It is assumed that the tooth to be treated before treatment has at least a partial concavity, and that an area including the concavity and some other areas are covered with pellicle and coloring components (organic components).

First, the first treatment agent serving as a cleansing agent is applied to the surface of the teeth to be treated (Step 1). The teeth to be treated to which the cleansing agent has been applied is left in that state for one minute. In this way, the cleansing agent dissolves and transforms the pellicle and coloring components covering the surface of the teeth to be treated. Further, the cleansing agent also dissolves the surface layer of the teeth beneath the layer of pellicle and coloring components.

Next, the cleansing agent on the surface of the teeth to be treated is wiped off by melamine foam (melamine sponge) (Step 2). The adhesion of the pellicle and coloring components on the surface of the teeth to be treated has been weakened by the treatment in Step 1. In addition, the melamine foam has a high ability to remove surface adhering substance by wiping. Therefore, the melamine foam absorbs the cleansing agent in a gel-like liquid in which the pellicle and coloring components are dissolved, and also strips off the pellicle and coloring components from the surface of the teeth to be treated. As a result, a clean enamel layer (hydroxyapatite) is exposed on the surface of the treated teeth including the recess. In addition, a part of the exposed surface layer of hydroxyapatite is ionized by the action of citric acid.

Next, the tooth surface treatment agent is applied to the tooth surface to be treated (step 3) that has already been cleaned in steps 1 and 2. The treated teeth to which the tooth surface treatment agent has been applied is left in that state for 3 minutes. The tooth surface treatment agent contains supersaturated Ca²⁺ ions and phosphate ions (one or more of H₂PO₄⁻, HPO₄²⁻, and PO₄³⁻) in which hydroxyapatite powder has been dissolved. No precipitation or agglomeration of hydroxyapatite particles can be observed in the aforementioned tooth surface treatment agent. The hydroxyapatite particles that have not been dissolved are considered to have been dispersed in the gel-like liquid as stable nanoparticles due to adsorption of citric acid on their surface. While the above-mentioned tooth surface treatment agent is left applied, crystals of hydroxyapatite and the like are formed on the surface of the teeth to be treated, including the concave areas. In other words, a layer of crystals of hydroxyapatite and the like will cover the surface of the teeth including the recesses.

Next, the tooth surface treatment agent is removed from the surface of the teeth after the treatment in step 3 (step 4). The removal of the tooth surface treatment agent can be done by simply rinsing with water, but it is preferable to rinse with water after wiping the teeth with a wipe material. If the wipe material is rubbed hard during wiping, the layer consisting of crystals such as hydroxyapatite may be peeled off. Water-impregnated melamine foam is suitable as a wipe material because it can remove the tooth surface treatment agent (which is difficult to remove by rinsing alone) by wiping lightly as if tracing the tooth surface. The excess Ca²⁺ ions on the tooth surface and crystals that have not been settled on the tooth surface can also be removed by the wiping using water-impregnated melamine foam.

By the above steps 1 to 4, the microscopic recesses on the tooth surface are covered with a layer composed of crystals such as hydroxyapatite. If the recesses are large in aspect ratio, the recesses will have cavities inside as a result of their upper portions being covered with a layer consisting of hydroxyapatite or other crystals. Therefore, it is desirable to strengthen the layer consisting of crystals such as hydroxyapatite covering the recesses, in order to maintain the recesses restored for a long period of time.

Fluoride treatment with the aforementioned fluoride treatment agent (gel-like liquid containing sodium fluoride) is extremely effective in strengthening the layer consisting of crystals such as hydroxyapatite formed in Step 3. Therefore, following the treatment in Step 4, the fluoride treatment agent is applied to the surface of the teeth to be treated (Step 5). Subsequently, the treated teeth coated with the fluoride treatment agent is then left in that state for 3 minutes. This strengthens the layer consisting of hydroxyapatite and other crystals in terms of both hardness and acid resistance. The strengthening of the layer consisting of hydroxyapatite and other crystals is presumably due to the fact that the F-ion contained in the fluoride treatment agent promotes the growth of hydroxyapatite crystals and the formation of new crystal nuclei. After the fluoride treatment, the fluoride treatment agent is removed in the same manner as in step 4 (step 6). Nevertheless, steps 5 and 6 are not shown in FIG. 1 because they are optional steps.

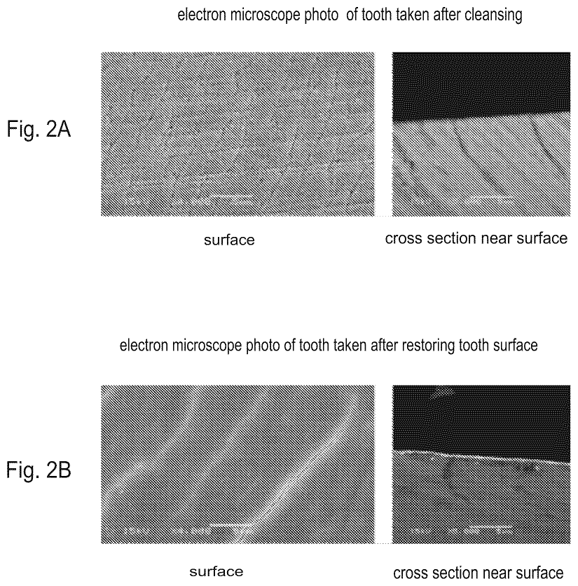

FIG. 2B shows an electron micrograph (reflected electron image) of the enamel surface layer of test teeth in which the above steps 1 to 6 were performed according to example 1. FIG. 2A is an electron micrograph (reflection electron image) of the enamel surface layer of test teeth that were only cleaned according to steps 1 to 2 (using treatment agent 1 of example 1). In the reflection electron image shown in FIG. 2B, the enamel surface layer is whitish (higher intensity of reflection electrons) compared to the reflection electron image in FIG. 2A. In the reflection electron image of FIG. 2B, the intensity of reflected electrons is higher in the groove-shaped concave area. This indicates that a new layer with a higher density than the enamel itself has been formed by performing steps 1 to 6.

In addition, the Micro-Vickers hardness of the test teeth having performed the above steps 1 to 6 have increased from 20 HV of the original test teeth to 170 HV. On the other hand, when the original test teeth were treated with only the fluoride treatment described in steps 5 to 6, the Micro-Vickers hardness was 90 I-IV. In other words, the treatments of the above steps 1 to 6 were found to be effective in strengthening the tooth structure of the entire tooth surface in addition to restoring the tooth surface recesses of the treated teeth.

The tooth surface treatment agent and tooth surface treatment kit of the present invention can be used for restoration of fine recesses on the tooth surface. Since the tooth surface treatment agent and tooth surface treatment kit of the present invention can restore the fine recesses on the tooth surface by forming a layer covering the entire tooth surface, the tooth surface can be strengthened by the film thus formed. The tooth surface treatment agent and tooth surface treatment kit can also produce a whitening effect since they form a layer that uniformly covers the entire tooth surface.

Although the embodiments of the present invention have been described above, the invention is not limited to the above embodiments, and various variations are possible within the scope of the claims and the technical ideas described in the specification and drawings.

1. A tooth surface treatment agent characterized by including:
a liquid with a pH of 1.0 to 3.0 containing an organic acid; and
hydroxyapatite particles with a particle diameter of 200 nm or less.

2. The tooth surface treatment agent according to claim 1, wherein the organic acid is a carboxylic acid having two carboxy groups.

3. The tooth surface treatment agent according to claim 1, wherein the hydroxyapatite particles are particles manufactured from biologically derived raw materials with a particle diameter of 100 nm or less.

4. A tooth surface treatment kit characterized by including:
a liquid first treatment agent containing an organic acid with a pH of 1.0 to 3.0, which is accommodated in a first container;
a second treatment agent of hydroxyapatite particles with a particle diameter of 200 nm or less, which is accommodated in a second container different from the first container;
wherein
the first treatment agent and the second treatment agent are mixed together to prepare a tooth surface treatment agent which is applied to the tooth surface.

5. A tooth surface treatment kit characterized by including:
a first liquid treatment agent containing an organic acid and having a pH of 1.0 to 3.0, which is accommodated in a first container; and
a second treatment agent of hydroxyapatite particles with a particle size of 200 nm or less, which is accommodated in a second container different from the first container;
wherein
the tooth surface is cleaned by applying the first treatment agent to the tooth surface;
the first treatment agent and the second treatment agent are mixed together to prepare a tooth surface treatment agent which is applied to the tooth surface.

6. The tooth surface treatment kit according to claim 4, further including a liquid fluoride treatment agent containing at least one of sodium fluoride, calcium fluoride, and stannous fluoride, which is accommodated in a third container different from the first and second containers,
wherein the fluoride treatment agent is applied to the tooth surface, after the tooth surface treatment agent applied to the tooth surface has been removed.

7. The tooth surface treatment kit according to any one of claim 4, wherein the organic acid is a carboxylic acid having two carboxy groups.

8. The tooth surface treatment kit according to any one of claim 4, wherein the hydroxyapatite particles are hydroxyapatite particles manufactured from biologically derived raw materials with a particle diameter of 100 nm or less.
